(19) United States
(12) Patent Application Publication
(10) Pub. No.: US 2020/0292544 A1
(43) Pub. Date: Sep. 17, 2020

(54) Protein Stability-based Small Molecule Biosensors and Methods

(71) Applicant: President and Fellows of Harvard College, Cambridge, MA (US)

(72) Inventors: George M. Church, Brookline, MA (US); Daniel J. Mandell, Brookline, MA (US); Justin Feng, Cambridge, MA (US)

(73) Assignees: President and Fellows of Harvard College, Cambridge, MA (US); President and Fellows of Harvard College, Cambridge, MA (US)

(21) Appl. No.: 16/083,935

(22) PCT Filed: Mar. 9, 2017
(86) PCT No.: PCT/US17/21526

§ 371 Date: Sep. 11, 2018

Related U.S. Application Data
(60) Provisional application No. 62/306,715, filed on Mar. 11, 2016.
(60) Provisional application No. 62/403,258, filed on Oct. 3, 2016.

(51) Int. Cl. G01N 33/566; C07K 14/435

(52) U.S. Cl. CPC G01N 33/566; C07K 2319/70; C07K 2319/80; C07K 14/435

(57) ABSTRACT

The disclosure provides a biosensor having a ligand binding domain (LBD) or its variant, wherein the stability of the LBD or its variant is conditioned on the presence of specific small molecule ligands, and wherein the LBD or its variant is fused to a reporter protein. The disclosure also provides a method of screening for small molecules that modulate protein stability.

### RELATED APPLICATION DATA

This application claims priority to U.S. Provisional Application No. 62/306,715 filed on Mar. 11, 2016 and to U.S. Provisional Application No. 62/403,258 filed on Oct. 3, 2016 which are hereby incorporated herein by reference in their entirety for all purposes.

### STATEMENT OF GOVERNMENT INTERESTS

This invention was made with government support under DE-FG02-02ER63445 awarded by the U.S. Department of Energy and under DGE1144152 awarded by the National Science Foundation. The government has certain rights in the invention.

### FIELD

The present invention relates in general to protein stability-based small molecule biosensors and methods.

### BACKGROUND

Small molecules play important roles across diverse biological processes, yet methods for detecting their abundance and effect on protein stability are lacking. Common methods for detecting proteins, such as genetic fusion to FRET reporters, N. Mochizuki et al., Nature 411, 1065 (Jun. 28, 2001), to peptides that ligate to chemical probes, G. Gaietta et al., Science 296, 503 (Apr. 19, 2002), or to protein complementation fragments, C. D. Hu, T. K. Kerppola, Nat Biotechnol 21, 539 (May, 2003), are typically inapplicable to the detection of small molecules.

Some metabolite binding proteins undergo dramatic conformational rearrangements upon complex formation with target molecules, so that genetic fusion to FRET pairs or labeling with environmentally sensitive dyes are natural routes to detection, R. Y. Tsien, S. A. Hires, Y. L. Zhu, Proc Natl Acad Sci USA 105, 4411 (Mar. 18, 2008). However, this approach is limited to the minority of well-characterized cases exhibiting allosteric transduction of small-molecule binding events.

Other techniques such as the SNAP, M. A. Brun, K. T. Tan, E. Nakata, M. J. Hinner, K. Johnsson, J Am Chem Soc 131, 5873 (Apr. 29, 2009), and HaloTag, G. V. Los et al., ACS Chem Biol 3, 373 (Jun. 20, 2008), methods involve covalent fusion of target analogs to environmentally-sensitive fluorescent tags that self-label to reporter protein complexes. High target concentrations then compete away the lower affinity analogs causing a shift in reporter fluorescence. There is a great need for protein stability-based small molecule biosensors and methods that enable high-throughput evaluation of the effect of small molecules on the stability of proteins or their variants for drug discovery and optimization.

### SUMMARY

The present disclosure provides protein stability-based small molecule biosensors engineered from conditionally stable ligand-binding domains (LBDs). The LBDs can include proteins, enzymes, engineered monoclonal antibodies (mAbs), or mAb fragments such as FAbs, scFvs, or nanobodies. These biosensors are prepared by using a general, modular approach to engineer proteins using the degree of protein stability conditioned upon binding to specific small molecule ligands, whereas the conditional protein stability and the abundance of the specific small molecule ligands are coupled to the activity and/or function of a reporter protein that can be detected by methods including fluorescence, catalysis, signaling, gene transcription or protein expression (FIG. 1).

The present disclosure provides biosensors including an LBD or its variant/mutant fused to a reporter protein. These fusion constructs can be prepared in two general schemes.

In the first scheme, mutations are introduced to a ligand binding domain (LBD) that render the LBD conditionally stable in the presence of the cognate small molecule ligand. The LBD is directly fused to a reporter protein. In the absence of the small molecule ligand, the LBD-reporter fusion protein is unstable and gets aggregated or degraded in prokaryotic cells, or aggregated or degraded by the ubiquitin-proteasome system (UPS) in eukaryotic cells. In the presence of a stabilizing target/small molecule ligand, the fusion protein is stabilized and its activity and/or function can be detected.

In the second scheme, a wild-type or LBD variant/mutant of the first scheme is fused to a RNA polymerase or a transcription factor (TF) that can include a DNA-binding domain and transcriptional activation/repression domain that activates/represses expression of a reporter gene. Fusion of a conditionally stable ligand-binding domain with a RNA polymerase generates a conditionally stable fusion protein. In the absence of the small molecule ligand, the fusion protein is unstable and gets aggregated or degraded in prokaryotic cells, or aggregated or degraded by the ubiquitin-proteasome system (UPS) in eukaryotic cells, thereby the downstream reporter gene expression is abrogated. In the presence of a stabilizing target/small molecule ligand, the fusion protein is stabilized, thereby coupling transcription of the downstream reporter gene to the abundance of the small molecule ligand as well as the degree of stability of the fusion protein. Fusion of a conditionally stable ligand-binding domain with a transcriptional activator generates a conditionally stable fusion protein. In the absence of the small molecule ligand, the fusion protein is unstable and gets aggregated or degraded in prokaryotic cells, or aggregated or degraded by the UPS in eukaryotic cells, thereby the downstream reporter gene expression is abrogated. In the presence of a stabilizing target/small molecule ligand, the fusion protein is stabilized, thereby coupling transcriptional activation of the downstream reporter gene to the abundance of the small molecule ligand as well as to the degree of stability of the fusion protein. When the conditionally stable ligand-binding domain is fused with a transcriptional repressor, the converse is true for reporter gene expression. Relative to the first scheme that uses a LBD fused directly to a reporter protein, the TF-based and polymerase-based systems of the second scheme should be more sensitive and have a wider dynamic range at the expense of a less rapid response time.

Several fusion constructs were created and tested. These constructs minimally encode a small molecule binding domain fused to a transcription factor, a polymerase, a single chain variable fragment (scFv), an enzyme or other reporter protein.

The present disclosure provides a biosensor including a ligand binding domain (LBD) or its variant, wherein the stability of the LBD or its variant is conditioned on the presence of specific small molecule ligands, and wherein the LBD or its variant is fused to a reporter protein. The reporter protein of the disclosure includes a fluorescent protein, a polymerase, a transcription factor (TF), an enzyme, a signaling protein, or a functional protein. The TF of the disclosure includes a transcriptional activator or repressor. The biosensor of the disclosure further includes elements suitable for screening of specific small molecule ligands that bind and stabilize/destabilize the LBD or its variant in prokaryotic or eukaryotic cells. The disclosure provides the biosensor wherein in the absence of a stabilizing ligand the LBD-reporter or the LBD variant-reporter fusion is unstable and degraded or targeted to an inclusion body in prokaryotic cells or eukaryotic cells, thereby preventing the reporter protein from carrying out its function. The disclosure provides the biosensor wherein in the presence of a stabilizing ligand the LBD-reporter or the LBD variant-reporter fusion is stabilized in prokaryotic cells or eukaryotic cells, thereby the reporter protein carries out its function.

The present disclosure provides that the transcriptional repressor includes a Lacl, a LexA, a 933W, or a cI from Lambda phage. The disclosure provides the biosensor wherein in the absence of a stabilizing ligand the LBD-repressor fusion or the LBD variant-repressor fusion is unstable and aggregated or degraded in prokaryotic cells, or aggregated or degraded by the UPS in eukaryotic cells, thereby activating transcription of a reporter gene. The disclosure provides the biosensor wherein in the presence of a stabilizing ligand the LBD-repressor fusion or the LBD variant-repressor fusion is stabilized in the prokaryotic or eukaryotic cells, thereby abrogating transcription of a reporter gene. The disclosure provides the biosensor wherein the LBD or its variant is fused to a RNA polymerase. The disclosure provides the biosensor wherein the LBD or its variant is fused to a RNA polymerase omega subunit and a DNA binding domain (DBD). The DNA binding domain of the disclosure is a Lacl, a LexA, a 933W, or a cI from Lambda phage that can activate transcription using the DBD cognate promoter. The disclosure provides the biosensor wherein the LBD or its variant is fused to a sigma factor as a sequence-specific activator. The TF of the disclosure provides a DNA-binding domain and transcriptional activation domain. The disclosure provides the biosensor wherein ligand-induced stabilization of the LBD-TF fusion or the LBD variant-TF fusion activates or represses expression of a reporter gene. The disclosure provides the biosensor wherein addition of a cognate ligand stabilizes the LBD-TF fusion or the LBD variant-TF fusion and increases in vivo levels of the TF, thus coupling transcriptional activation to the level of the small molecule ligand. The disclosure provides that the LBD and the reporter protein are genetically fused. The disclosure further provides that the LBD and the reporter protein are fused together post-translationally. The LBD of the disclosure includes proteins, enzymes, engineered monoclonal antibodies (mAbs), or mAb fragments, FAbs, scFvs or nanobodies. The LBD enzymes of the disclosure includes EEF1A1, GAPDH or PKM2.

The present disclosure further provides a cell-free biosensing system including a biosensor that includes a ligand binding domain (LBD) or its variant, wherein the stability of the LBD or its variant is conditioned on the presence of specific small molecule ligands, and wherein the LBD or its variant is fused to a reporter protein. The disclosure provides that the reporter protein includes a fluorescent protein, a transcription factor (TF), an enzyme, a signaling protein, or a functional protein. The disclosure further provides that the LBD, its variant and the reporter protein are purified or in vitro transcribed, translated and degraded using whole cell lysate from cells including bacteria, yeast, human, wheat germ or rabbit reticulocytes, or using purified transcription, translation and degradation components. The TF of the present disclosure includes a DNA-binding domain and a transcriptional activator or repressor. The disclosure provides that ligand-induced stabilization of the LBD-TF fusion or the LBD variant-TF fusion activates or represses expression of a reporter gene. The disclosure further provides that addition of a cognate ligand stabilizes the LBD-TF fusion or the LBD variant-TF fusion and increases levels of the TF, thus coupling transcriptional activation to the level of the small molecule ligand.

The present disclosure provides that the LBD or its variant is fused to the DNA-binding domain of Gal4 and a VP16 activation domain. The disclosure further provides that the LBD or its variant is fused to an RNA polymerase. The RNA polymerase of the disclosure includes T7, T3 or SP6 RNA polymerases. The LBD of the disclosure includes proteins, enzymes, engineered monoclonal antibodies (mAbs), or mAb fragments, FAbs, scFvs or nanobodies. The LBD enzyme includes EEF1A1, GAPDH or PKM2.

The present disclosure provides a method of screening protein stabilizing small molecule ligands including contacting a sample suspected of containing the small molecule ligand with a biosensor comprising a ligand binding domain (LBD) or its variant, wherein the stability of the LBD or its variant is conditioned on the presence of specific small molecule ligands, and wherein the LBD or its variant is fused to a reporter protein, detecting the amount of the reporter protein wherein the amount of the reporter protein is dependent on the stability of the LBD or its variant, and selecting the small molecule ligand that stabilizes the LBD or its variant. The disclosure provides a reporter protein that includes a fluorescent protein, a RNA polymerase, a transcription factor (TF), an enzyme, a signaling protein, or a functional protein. The disclosure further provides that a library of mutational LBD variants is created. The disclosure provides that the sequence of the mutational LBD variant can be determined by sequencing. The disclosure further provides that the sequencing is next-generation sequencing or Sanger sequencing. The disclosure provides a method that selects for ligand-LBD pairs showing stabilization or destabilization over control.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

It should be appreciated by those persons having ordinary skill in the art(s) to which the present disclosure relates that any of the features described herein in respect of any particular aspect and/or embodiment of the present disclosure can be combined with one or more of any of the other features of any other aspects and/or embodiments of the present disclosure described herein, with modifications as appropriate to ensure compatibility of the combinations. Such combinations are considered to be part of the present disclosure contemplated by this disclosure.

It is to be understood that both the foregoing general description and the following detailed description are exemplary and explanatory only and are not restrictive of the disclosure as claimed. Other embodiments will be apparent to those skilled in the art from consideration of the specification and practice of the disclosure disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present disclosure will be more fully understood from the following detailed description of illustrative embodiments taken in conjunction with the accompanying drawings in which:

FIG. 1 is an illustration of a protein-based in vivo small molecule biosensor. A natural or engineered protein that binds a small molecule target is converted to a sensor by introducing destabilizing mutations. Destabilization of the sensor due to low target abundance induces degradation or aggregation of the entire construct, creating a modular biosensor for the target.

FIG. 2 is an illustration of a process for constructing protein-based small molecule biosensors by error-prone PCR and FACS sorting.

FIGS. 3A-C depict illustrations of antibody scaffolds as ligand-binding domains in protein-based biosensors. FIG. 3A shows multiple rounds of FACS performed on libraries of scFv mutants can select for variants that are conditionally stable on binding a target ligand. Key target binding residues are shown as green circles and conditionally destabilizing mutations are shown as red Xs. FIG. 3B shows a scFv that has been rendered unstable can become conditionally stable on a target by porting known ligand-binding sequences from another scFv. Alternatively, ligand-binding sequences can be determined by immunization, phage display, yeast display, in vitro screening, computational protein design, or other methods. FIG. 3C shows a scFv that binds a target can be rendered conditionally stable on the target by porting destabilizing mutations from another destabilized or conditionally stable scFv. In all cases, while scFvs are shown, the same techniques can be applied across full antibodies and antibody fragments, including mAbs, FAbs and nanobodies.

FIG. 4A-D depict results of experiments directed to the ability of an scFvs sensor to induce yEGFP expression in yeast.

FIG. 5A-B depict the results of fold induction of GFP fluorescence levels normalized to mCherry levels over increasing concentrations of digoxin (left) and progesterone (right) in E. coli.

FIG. 6. depicts the results of luminescence levels of LBD biosensors fused to various reporters and expressed in vitro in rabbit reticulocyte lysates.

### DETAILED DESCRIPTION

Embodiments of the present disclosure are directed to a general, modular approach to engineer protein-based biosensors that utilize the degree of protein stability conditioned upon binding to specific small molecule ligands, whereas the conditional protein stability and the abundance of the specific small molecule ligands are coupled to the activity and/or function of a reporter protein that can be detected by methods including fluorescence, catalysis, signaling, gene transcription or protein expression. The generalizability of this disclosure arises from its basis on two pervasive biophysical principles: molecular recognition and protein folding.

Embodiments of the present disclosure are directed to protein stability-based small molecule biosensors engineered from conditionally stable ligand-binding domains (LBDs). The LBDs can include proteins, enzymes, engineered monoclonal antibodies (mAbs), or mAb fragments such as FAbs, scFvs, or nanobodies.

According to one aspect, sensors are constructed in two exemplary ways. In the first method, mutations are introduced to a ligand binding domain (LBD) that render the LBD conditionally stable upon the presence of the cognate small molecule ligand. The LBD is directly fused to a reporter protein. In the absence of the small molecule ligand, the LBD-reporter fusion protein is unstable and gets aggregated or degraded in prokaryotic cells, or aggregated or degraded by the UPS in eukaryotic cells. In the presence of stabilizing target/small molecule ligand, the fusion protein is stabilized and its activity and/or function can be detected.

In the second method, a wild-type or LBD variant/mutant of the first scheme is fused to a transcription factor (TF) that can include a DNA-binding domain and transcriptional activation/repression domain that activates/represses expression of a reporter gene. Fusion of a conditionally stable ligand-binding domain with a transcriptional activator generates a destabilized fusion protein. In the absence of the small molecule ligand, the fusion protein is unstable and gets aggregated or degraded in prokaryotic cells, or aggregated or degraded by the UPS in eukaryotic cells, thereby the downstream reporter gene expression is abrogated. In the presence of stabilizing target/small molecule ligand, the fusion protein is stabilized, thereby coupling transcriptional activation of the downstream reporter gene to the abundance of the small molecule ligand as well as to the degree of stability of the fusion protein. When the conditionally stable ligand-binding domain is fused with a transcriptional repressor, the converse is true for reporter gene expression. Relative to the first scheme that uses a LBD fused directly to a reporter protein, the TF-based system of the second scheme should be more sensitive and have a wider dynamic range at the expense of a less rapid response time.

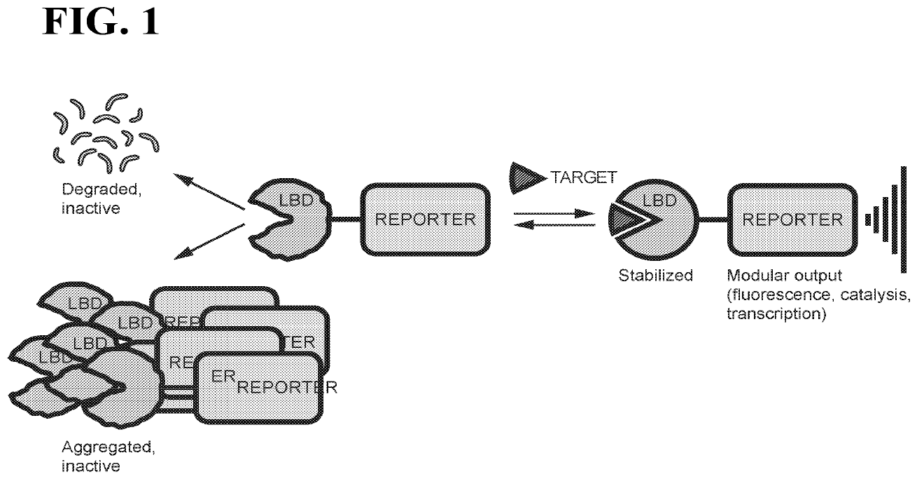

To build a sensor by the first method, mutations are introduced to an existing LBD that binds the target, which is genetically fused to a reporter protein (FIG. 1). The construct can be inducibly or constitutively expressed. In the absence of its small molecule target, the LBD is destabilized and tagged for degradation, thus abrogating the reporter signal. Introduction of the target small molecule stabilizes the LBD and rescues reporter function. As a result, the level of reporter activity depends on local target concentration. In principle any genetically encodable polypeptide can serve as the modular reporter, and the only requirement for construction is the existence of a LBD.

The starting LBD sequence can either come from an engineered or naturally occurring binding protein, or can be designed in silico. In silico designs can produce physiologically orthogonal binding proteins for ligands lacking a suitable natural LBD. See C. E. Tinberg et al., Nature 501, 212 (Sep. 12, 2013). Thus, sensors built using this approach have few prerequisites, and once engineered can be fused to various reporters in a modular fashion, for example switching from fluorescence for screening and imaging to a transcription factor for regulating a metabolic pathway. Further, since prokaryotic organisms also degrade unstable proteins and/or target them to inclusion bodies, the system is likely to be portable to prokaryotic cells.

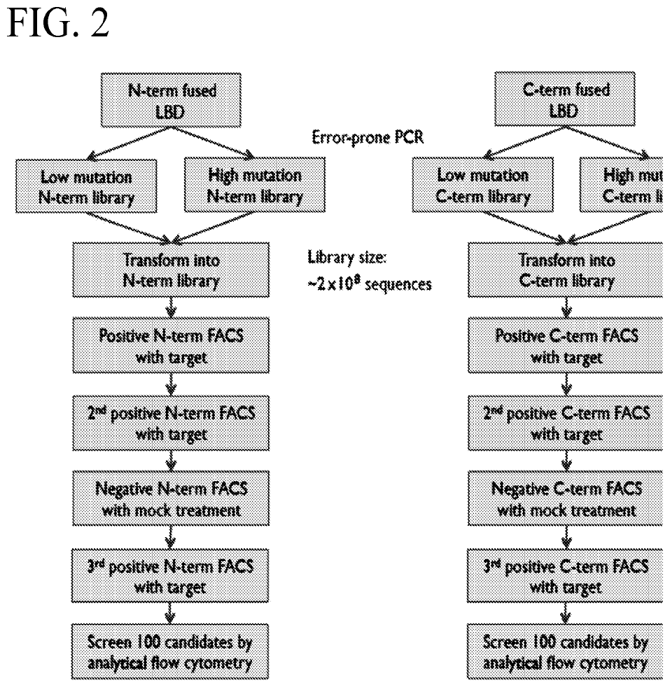

As proof of principle, a LBD fused to a GFP reporter is constructed and subjected to error-prone PCR followed by multiple rounds of FACS sorting (FIG. 2).

Exemplary construction of LBDs and their variants in yeast such as DIG10.3 variants for conditionally stable digoxigenin and progesterone LBDs; sorting and screening of digoxigenin and progesterone LBD fusions to yeGFP; reporter plasmid construction and integration; Gal4-DIG10.3-VP and mutant plasmid construction; Gal4-DIG10.3-VP progesterone variant construction; and Gal4-DIG10.3-VP16 error-prone library construction and selections have been described in U.S. application Ser. No. 14/993,509 filed on Jan. 12, 2016 and WO Patent Application No. PCT/US16/13005, filed Jan. 12, 2016, each of which are hereby incorporated by reference in its entirety. These yeast biosensor constructs can be modified for a prokaryotic cell such as an E. coli cell using methods known to one skilled in the art. E. coli-specific transcription factor-promoter pairs are constructed for screening E. coli specific conditionally destabilizing mutants.

Exemplary computational model for constructing LBD fusion constructs and ligand specificity assays, kinetic and reporter assays, yeast spotting assays and screening protocols in K562 cells lines have been described in U.S. application Ser. No. 14/993,509 filed on Jan. 12, 2016 and WO Patent Application No. PCT/US16/13005, filed Jan. 12, 2016, each of which are hereby incorporated by reference in its entirety. These assays and screening protocols can be readily modified for a prokaryotic cell such as an E. coli cell or in a cell-free system according to the present disclosure.

Cells according to the present disclosure include any cell into which LBD and fusion constructs can be introduced and expressed as described herein. It is to be understood that the basic concepts of the present disclosure described herein are not limited by cell type. Cells according to the present disclosure include eukaryotic cells, prokaryotic cells, animal cells, plant cells, fungal cells, archael cells, eubacterial cells and the like. Cells include eukaryotic cells such as yeast cells, plant cells, and animal cells.

Cell-free or in vitro biosensors and in vitro libraries can be generated according to the methods described herein. In vitro transcription and translation can be carried out by purified components or by whole cell lysate from cells including bacteria, yeast, human, wheat germ and rabbit reticulocytes.

The biosensors, as described in U.S. application Ser. No. 14/993,509 filed on Jan. 12, 2016 and WO Patent Application No. PCT/US16/13005, filed Jan. 12, 2016, each of which are hereby incorporated by reference in its entirety, can be ported into a cell-free system using previously characterized lysates from eukaryotic organisms such as yeast, rabbit reticulocyte, HeLa, bacteria and wheat germ. A variety of polymerase-promoter pairs to test the compatibility of the biosensors with in vitro transcription, translation and degradation in rabbit reticulocyte lysate are constructed. In an exemplary embodiment, the polymerase that transcribes the reporter signal is fused directly to the LBD, as opposed to fusing a TF that recruits an endogenous polymerase. As proof of principle, polymerases are fused directly to LBDs and tested in yeast for transcribing reporter signals.

The disclosure provides antibody scaffolds as starting points for biosensor construction. Monoclonal antibodies (mAbs) or fragments thereof including FAbs, scFvs, or nanobodies can be engineered to tightly bind target molecules. The mAbs and mAb fragments can be expressed in cells or cell-free systems as described herein. Framework mutations to the constant regions of antibody products may provide generalized destabilized scaffolds, so that sensors to new targets can be produced simply by grafting ligand recognizing elements such as variable loops onto destabilized antibodies or antibody fragments. Ligand-binding sequences may be known a priori, or may be determined from antibodies raised by immunization in animals or tissue culture, or by screening from phage display, yeast display, or in vitro methods. As proof of principle, a known anti-digoxin single chain variable fragment (scFv) is created in place of the digoxin-binding LBD in the yeast system as herein described, and mutants are created to remove the scFv disulfides or secretion signal peptides are added to the scFv to target it to organelles that permit disulfides to improve the stability of the scFv construct. In prokaryotic cells, the disulfide-forming cysteines are mutated to other standard amino acids including valine or alanine, or they are replaced with nonstandard or noncanonical amino acids including selenocysteine that can form covalent bonds including diselenide bonds that are not reduced in the prokaryotic cytoplasm as described in WO Patent Application No. PCT/US15/57780, filed Oct. 28, 2015, the contents of which are hereby incorporated by reference in its entirety, or the antibody or antibody fragment is targeted for periplasmic secretion to facilitate disulfide bond formation.

Vectors according to the present disclosure include those known in the art as being useful in expressing genetic material in a cell or cell-free system and would include regulators, promoters, nuclear localization signals (NLS), start codons, stop codons, a transgene etc., and any other genetic elements useful for integration and expression, as are known to those of skill in the art.

It is to be understood that the embodiments of the present disclosure which have been described are merely illustrative of some of the applications of the principles of the present disclosure. Numerous modifications may be made by those skilled in the art based upon the teachings presented herein without departing from the true spirit and scope of the disclosure. The contents of all references, patents and published patent applications cited throughout this application are hereby incorporated by reference in their entirety for all purposes.

The following examples are set forth as being representative of the present disclosure. These examples are not to be construed as limiting the scope of the disclosure as these and other equivalent embodiments will be apparent in view of the present disclosure, figures, tables, and accompanying claims.

### Example I

### Bacterial Hosts for Biosensor Development and Deployment

Prokaryotic organisms like bacteria are also popular hosts for bioproduction, and thus present an additional need for biosensor-driven optimization of enzymes and biosynthetic pathways. See Zhang, F., Carothers, J. M. & Keasling, J. D. Design of a dynamic sensor-regulator system for production of chemicals and fuels derived from fatty acids. Nat Biotechnol 30, 354-359, doi:10.1038/nbt.2149 (2012) and Tang, S. Y. & Cirino, P. C. Design and application of a mevalonate-responsive regulatory protein. Angewandte Chemie 50, 1084-1086, doi:10.1002/anie.201006083 (2011).

Prokaryotes lack the conserved ubiquitin-proteasome system (UPS) that is expected to be critical for protein stability-based biosensor function across eukaryotes. See Egeler, E. L., Urner, L. M., Rakhit, R., Liu, C. W. & Wandless, T. J. Ligand-switchable substrates for a ubiquitin-proteasome system. J Biol Chem 286, 31328-31336, doi:10.1074/jbc.M111.264101 (2011).

However, there is evidence that destabilized proteins fused to a GFP reporter are aggregated and targeted to inclusion bodies, which prevents GFP from folding and fluorescing. See Drew, D. et al. A scalable, GFP-based pipeline for membrane protein overexpression screening and purification. Protein Sci 14, 2011-2017, doi:10.1110/ps.051466205 (2005). Further, unstable proteins can be degraded by prokaryotic proteases like ClpX and Lon. Likewise, the prokaryotic biosensors of the present disclosure are constructed so that the destabilized LBD-reporter fusion is degraded, aggregated, or targeted to an inclusion body in the absence of the stabilizing target ligand, preventing the reporter protein from carrying out its function. In the presence of stabilizing target ligand the fusion is stabilized and the reporter protein carries out its function.

The reporter protein may be a fluorescent protein, transcription factor, enzyme, signaling protein, or other functional protein. It has been previously found that using transcription factors as reporters for biosensor stability amplifies the signal relative to direct fusion to a fluorescent reporter. To enable discovery and use of biosensors in E. coli with maximum dynamic range, in addition to our previously described direct GFP-LBD fusions, the LBDs are fused to a panel of transcription factors including LexA, LacI (see Gilbert, W. & Muller-Hill, B. Isolation of the lac repressor. Proc Natl Acad Sci USA 56, 1891-1898 (1966)), 933W (see Plunkett, G., 3rd, Rose, D. J., Durfee, T. J. & Blattner, F. R. Sequence of Shiga toxin 2 phage 933W from Escherichia coli O157:H7: Shiga toxin as a phage late-gene product. J Bacteriol 181, 1767-1778 (1999)), and cI from Lambda phage (see Sauer, R. T. DNA sequence of the bacteriophage gama cI gene. Nature 276, 301-302 (1978)). These E. coli transcription factors primarily function as repressors by binding to their cognate DNA promoter site and blocking transcription. In these cases, biosensor instability in the absence of ligand will cause degradation or aggregation of the construct, activating transcription of the reporter gene. Stability in the presence of the target ligand will abrogate expression of the reporter.

In order to obtain sensors that activate, rather than repress, gene expression in the presence of the target small molecule, the LBDs are genetically fused to the RNA polymerase omega subunit (see Minakhin, L. et al. Bacterial RNA polymerase subunit omega and eukaryotic RNA polymerase subunit RPB6 are sequence, structural, and functional homologs and promote RNA polymerase assembly. Proc Natl Acad Sci USA 98, 892-897, doi:10.1073/pnas.98.3.892 (2001)) and a DNA binding domain (DBD) such as LexA, LacI, 933W, and cI from Lambda phage to activate transcription using the DBD cognate promoter. Sigma factors as sequence-specific activators can be fused to the LBD biosensors. See Rhodius, V. A. et al. Design of orthogonal genetic switches based on a crosstalk map of sigmas, anti-sigmas, and promoters. Mol Syst Biol 9, 702, doi: 10.1038/msb.2013.58 (2013).

Libraries of LBD mutants can be screened for ligand-dependent stability in bacteria in the same fashion as described in U.S. application Ser. No. 14/993,509 filed on Jan. 12, 2016 and WO Patent Application No. PCT/US16/13005, filed Jan. 12, 2016, each of which are hereby incorporated by reference in its entirety. For example, a library of bacteria bearing mutational variants of an LBD fused to a fluorescent protein—or to a transcriptional regulator controlling expression of a fluorescent protein—can be subjected to fluorescence-activated cell sorting (FACS), iteratively sorting for the fluorescent population in the presence of the target ligand, and for the dark population in the absence of the target ligand. Constructs demonstrating conditional stability can then be used to read out small molecule abundance in vivo, e.g., to select for bacteria strains with higher production of the target. Thus bacterial hosts like E. coli can serve as chassis for both the development and the deployment of protein stability-based biosensors.

### Example II

### Cell-Free Biosensors Employing In Vitro Transcription, Translation and Degradation

All in vivo approaches to biosensors require that the targets are membrane permeable and non-toxic. Further, biosensor library size is limited by the transformation efficiency of the host organism. In vitro biosensors remove the requirements of target membrane permeability and non-toxicity, and in vitro libraries can surpass the tractable size of in vivo libraries by many orders of magnitude. In vitro transcription and translation can be carried out by purified components or by whole cell lysate from cells including bacteria, yeast, human, wheat germ and rabbit reticulocytes. In vitro transcription, translation and subsequent degradation of unstable proteins by the UPS has been demonstrated in rabbit reticulocyte lysate (Ristriani T. et al., A single-codon mutation converts HPV16 E6 oncoprotein into a potential tumor suppressor, which induces p53-dependent senescence of HPV-positive HeLa cervical cancer cells, Oncogene, 2009, Vol. 28(5):762-72, Epub 2008 Nov. 17).

To port the existing biosensors into a cell-free system, previously characterized lysates from eukaryotic organisms such as yeast, rabbit reticulocyte, HeLa, bacteria and wheat germ will be used. Endogenous transcription and translational machinery will be tested using previously designed yeast and human promoters, as described in U.S. application Ser. No. 14/993,509 filed on Jan. 12, 2016 and WO Patent Application No. PCT/US16/13005, filed Jan. 12, 2016, each of which are hereby incorporated by reference in its entirety. The use of exogenously added RNA polymerases for both the GFP-direct fusion and TF-biosensor constructs will be tested by expressing them under the promoters for T7, T3, and SP6 RNA polymerases and supplying the corresponding purified RNA polymerase to the cell-free system.

The reporters in the yeast TF-biosensors use the VP16 transcriptional activator to recruit endogenous transcriptional machinery to the Gal4 DNA binding site, thereby driving expression of an output signal gene downstream from the Gal4 binding site. In a cell-free system, it is possible that the endogenous translational machinery is not sufficiently recruited to detectably express the reporter gene using the existing Gal4/VP16 system. To address this possibility, alternative transcription factor systems will be tested in parallel. The LBDs will be genetically fused directly to RNA polymerases (T7, T3, and SP6). The reporter gene will be placed under the cognate promoter for each polymerase, and the performance of each polymerase/promoter pair will be quantified in terms of characteristics like dynamic range, dose response, and kinetics of activation and deactivation. The RNA polymerase used to express the LBD fusion may be orthogonal to the RNA polymerase used to express the reporter gene.

To screen for new biosensors, libraries of LBD variant DNA can be in vitro transcribed into mRNA, which is translated into proteins, all using transcription and translation machinery from the lysate, or optionally supplemented with additional polymerases, ribosomes or other transcriptional or translational machinery. LBD variants function in successful biosensors if they meet the following screening criteria:

a. In the absence of the target ligand they are substantially degraded by the UPS or proteases existing in the lysate (or in supplemented variants thereof), or are otherwise substantially aggregated to prevent the reporter from carrying out its function,
b. In the presence of target ligand they are not degraded or aggregated (or are degraded or aggregated substantially less)

The DNA sequence of LBDs in successful biosensors can be determined by multiple methods, including mRNA display and ribosome display. See Lipovsek, D. & Pluckthun, A. In-vitro protein evolution by ribosome display and mRNA display. J Immunol Methods 290, 51-67, doi:10.1016/j.jim.2004.04.008 (2004). With a system employing these methods, the mRNA of LBD variants are physically associated with the LBD variant proteins, and the mRNA of LBD variants passing the screen is reverse transcribed to produce cDNA, and then the cDNA can be read by techniques such as Sanger or next-generation sequencing. Functional biosensors can also be enriched by using immunoprecipitation or pull-downs for standard sequence tags fused to the biosensor constructs prior to sequencing. The standard tags include Myc, Flag, poly-His, V5 and others.

Conditionally destabilized biosensors were engineered in cell-free systems such as lysates according to certain embodiments herein disclosed. Plasmids were created each containing a ligand binding domain (LBD) fused to T7 RNA polymerase or NanoLuc reporter. The RNA polymerase reporters drive expression of a T7 RNA polymerase responsive firefly luciferase reporter on a separate plasmid. The plasmids were added at 1 ng each to 25 uL of rabbit reticulocyte lysate and incubated for 1.5 hours at 30° C. NanoLuc direct fusions were diluted 1 uL of lysate into 7 uL PBS, and subsequently added to 100 uL of NanoGlo reagent (Promega) and immediately analyzed for luminescence levels. T3 and T7 RNA polymerase fusions were diluted 2.5 uL of lysate into 50 uL of firefly luciferase reagent buffer (Promega) and immediately analyzed for luminescence levels.

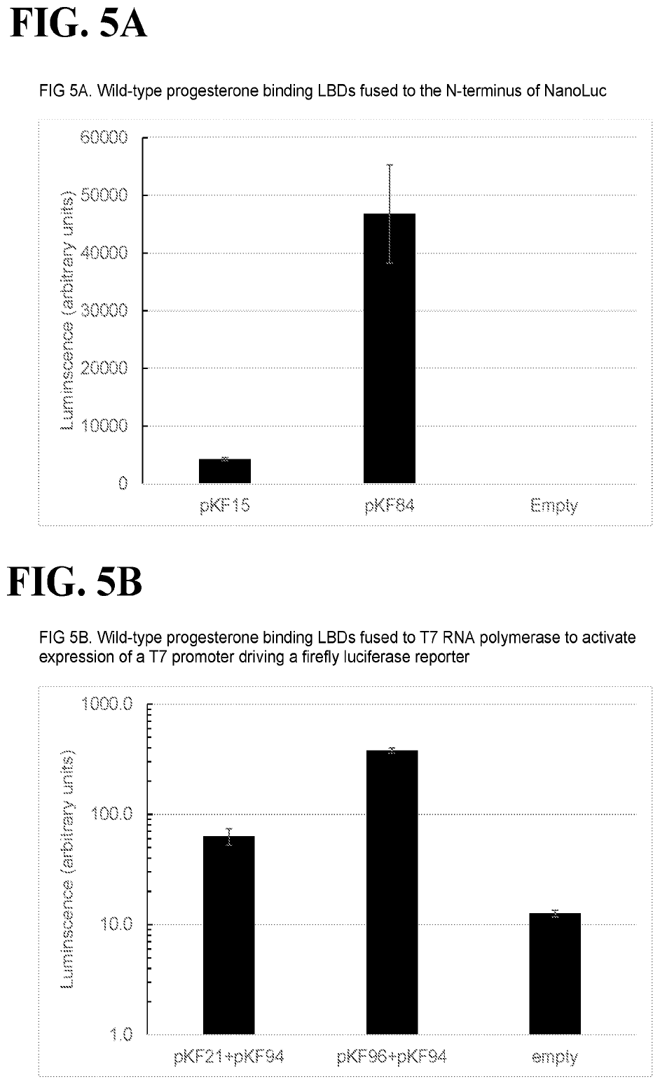

FIG. 5A and FIG. 5B. depict the results of luminescence levels of LBD biosensors fused to various reporters and expressed in vitro in rabbit reticulocyte lysates. The wild-type progesterone binding LBD (PRO₀ LBD) was fused to the N-terminus of NanoLuc (FIG. 5A) or to T7 RNA polymerase to activate expression of a T7 promoter driving a firefly luciferase reporter (FIG. 5B). pKF15 is the direct fusion of the progesterone binding LBD to NanoLuc, pKF84 is a positive control with NanoLuc alone, and “empty” indicates a negative control without any luciferase expression. pKF94 is the T7 activated firefly luciferase reporter plasmid. pKF21 is the progesterone binding LBD fused to T7 RNA polymerase. pKF96 is the T7 RNA polymerase alone, used as a positive control.

### Example III

### Antibody Scaffolds as Starting Points for Biosensor Construction

For some small molecule targets a known LBD may not exist, may interfere with cellular processes, or may bind the target with insufficient affinity. Monoclonal antibodies (mAbs) or fragments thereof including FAbs, scFvs, or nanobodies can be engineered to tightly bind target molecules. mAbs and mAb fragments can be expressed in mammalian cells, plants and yeast. They may also be expressed in E. coli by periplasmic secretion, by oxidizing the cytosol, or by replacing disulfide forming cysteines with other standard, noncanonical or nonstandard amino acids as described in WO Patent Application No. PCT/US15/57780, filed Oct. 28, 2015, the contents of which are hereby incorporated by reference in its entirety. Framework mutations to the non-ligand-binding regions of antibody products may provide generalized destabilized scaffolds, so that sensors to new targets can be produced simply by grafting ligand recognizing elements such as variable loops onto destabilized antibodies or antibody fragments. Ligand-binding sequences may be known a priori, or may be determined from antibodies raised by immunization in animals or tissue culture, or by screening from phage display (see Smith, G. P. Filamentous fusion phage: novel expression vectors that display cloned antigens on the virion surface. Science 228, 1315-1317 (1985)), yeast display (see Boder, E. T. & Wittrup, K. D. Yeast surface display for screening combinatorial polypeptide libraries. Nat Biotechnol 15, 553-557, doi:10.1038/nbt0697-553 (1997)), or in vitro methods (see He, M. et al. Selection of a human anti-progesterone antibody fragment from a transgenic mouse library by ARM ribosome display. J Immunol Methods 231, 105-117 (1999)). Thus antibody-based biosensors will be constructed through three general strategies:

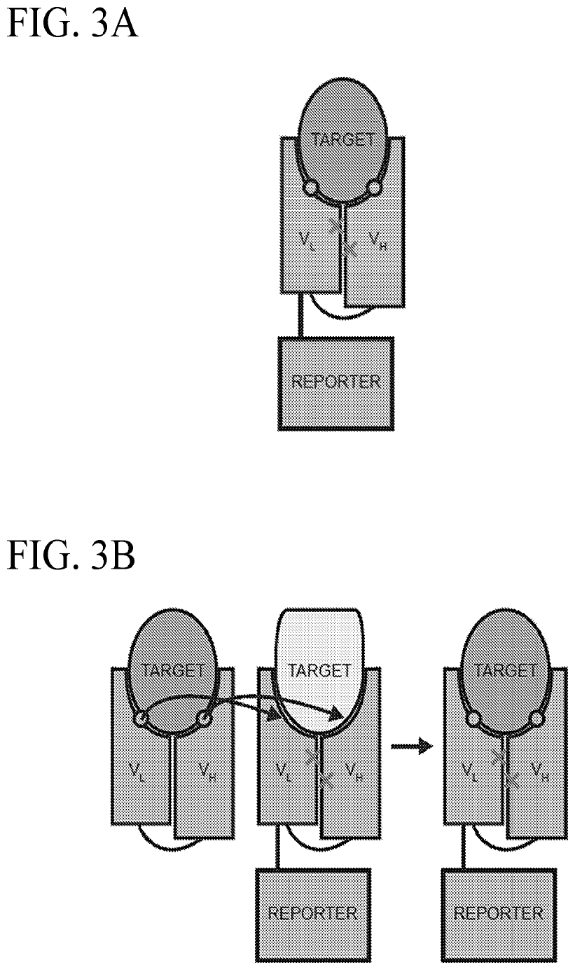

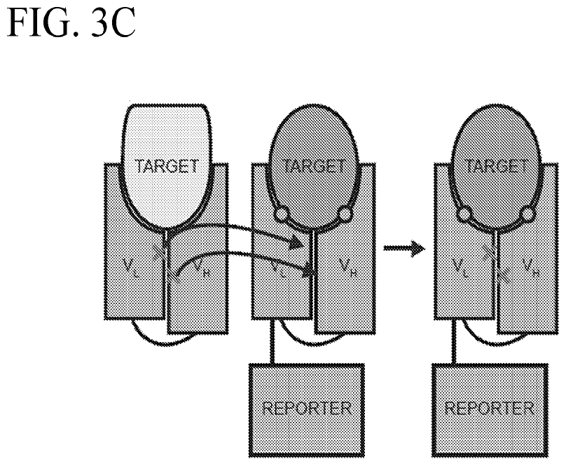

a. Using an existing antibody or antibody fragment for a target fused to a reporter (e.g., a fluorescent protein), mutagenize the antibody or antibody fragment and select or screen (e.g., by FACS) to discover mutations that render the antibody or antibody fragment conditionally stable on the target. This strategy has been described in eLife publication (see Feng, J. et al. A general strategy to construct small molecule biosensors in eukaryotes. eLife 4, doi:10.7554/eLife.10606 (2015)), for treating the antibody or antibody fragment as the LBD. This strategy is illustrated in FIG. 3A. The same strategy has also been described in U.S. application Ser. No. 14/993,509 filed on Jan. 12, 2016 and WO Patent Application No. PCT/US16/13005, filed Jan. 12, 2016, each of which are hereby incorporated by reference in its entirety.

b. Using a destabilized or conditionally stable antibody or antibody fragment as in (a), modify the ligand-binding regions to confer conditional stability from a different small molecule target. The sequence for the ligand-binding region can be obtained by immunization, by grafting a known ligand-binding sequence from another antibody or antibody fragment (see Jung, S. & Pluckthun, A. Improving in vivo folding and stability of a single-chain Fv antibody fragment by loop grafting. Protein Eng 10, 959-966 (1997)), by screening using techniques like phage display or yeast display, or by computational protein design (see Weitzner, B. D., Kuroda, D., Marze, N., Xu, J. & Gray, J. J. Blind prediction performance of RosettaAntibody 3.0: grafting, relaxation, kinematic loop modeling, and full CDR optimization. Proteins 82, 1611-1623, doi:10.1002/prot.24534 (2014)). This strategy is illustrated in FIG. 3B.

c. Starting with an antibody or antibody fragment that binds a given target, introduce framework mutations to non-ligand-binding sequences known in the field or discovered as in (a) for a different target (or from a different antibody or antibody fragment) while keeping the ligand-binding sequence constant, to confer conditional stability for the given target. This strategy is illustrated in FIG. 3C.

Cysteine-free digoxin-binding scFvs with expression in yeast were prepared. A cysteine-free scFv capable of binding digoxin was engineered by fusing V_{H} and V_{L} fragments with a flexible linker and grafting digoxin binding loops into the complementary determining regions of the scFv scaffold, resulting in sequence A2. The V_{H} sequence is MEVQLLESGGGLVQPGGSLRLSAAASGFTFSTFSMNWVRQAPGKGLEWVSYISRTSK TIYYADSVKGRFTISRDNSKNTLYLQMNSLRAEDTAVYYVARGRFFDYWGQGTLVT VSS. The V_{L} sequence is DIQMTQSPSSLSASVGDRVTITVRASQSISSYLNWYQQKPGEAPKLLIYSASVLQSGYP SRFSGSGSGTDFTLTISSLQPEDFATYYAQQSVMIPMTFGQGTKVETKR. The linker sequence is GGGGSGGGGSGGGGS. The following additional sequences were used.

Digoxin VH CDR1: NYWLG
Digoxin VH CDR2: DIYSGGGYTNHNEKFKG
Digoxin VH CDR3: SGPYDYDEVY
Digoxin VL CDR1: RASQDIGSSLN
Digoxin VL CDR2: ATSSLDS
Digoxin VL CDR3: LQYASSPWT.

The sequence for A2 is MEVQLLESGGGLVQPGGSLRLSAAASAYSLTNYWLGVRQAPGKGLEWVSDIYSGGG YTNHNEKFKGRFTISRDNSKNTLYLQMNSLRAEDTAAYYVARSGPYDYDEVYWGQ GTLVTVSSGGGGSGGGGSGGGGSDIQMTQSPSSLSASVGDRVTITVRASQDIGSSLN WYQQKPGEAPKLLIYATSSLDSGVPSRFSGSGSGTDFTLTISSLQPEDFATYYALQYAS SPWTFGQGTKVETKR.

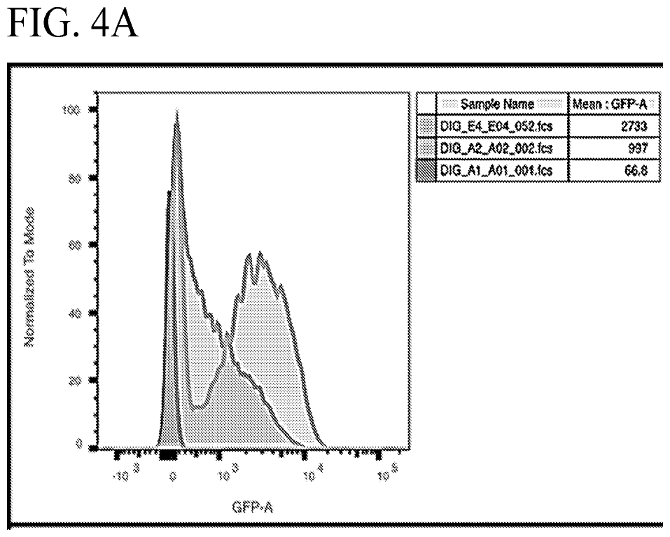

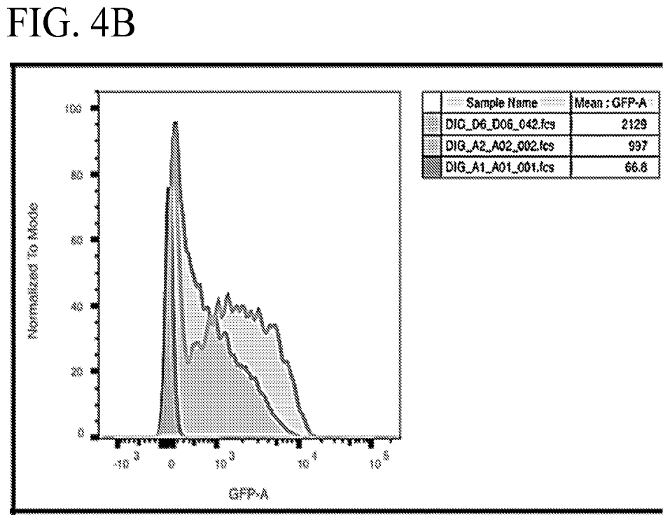

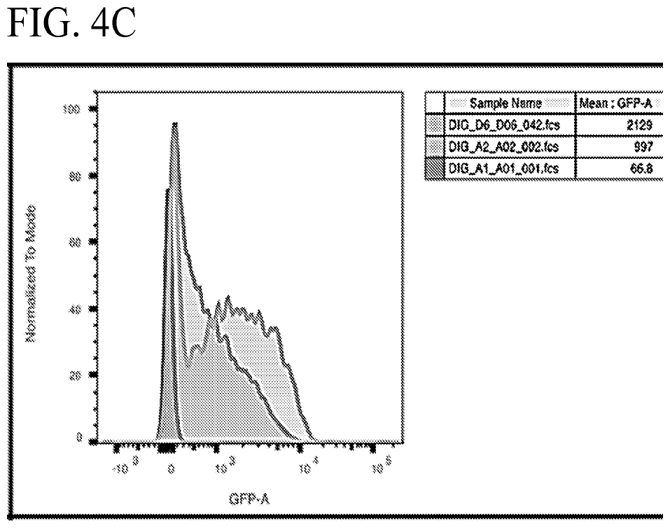

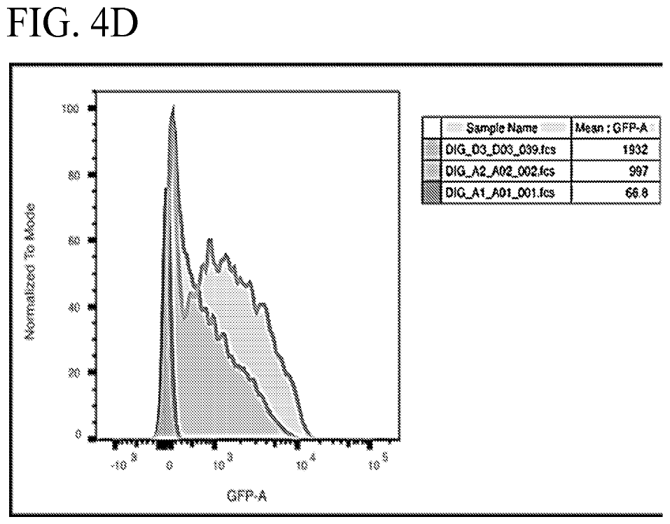

The scFv was placed into the biosensor construct as a Gal4-scFv-VP16 fusion and the construct was assayed for expression by ability to induce yEGFP expression in yeast. The starting sequence expressed poorly in yeast as indicated by analytical flow cytometry. Through error-prone PCR to the scFv coding sequence and FACS sorting, variants were engineered with strong expression in yeast as analyzed by flow cytometry. Variants with mutations to the scFv sequence A2 within the biosensor constructs analyzed by flow cytometry are as follows, with the cytometry data shown in FIG. 4A-D.
DIG_A1_A01_001: untransformed cells;
DIG_A2_A02_002: starting sequence;
DIG_E4_E04_052: starting sequence A2 with mutations F68S, Y80H, Q111L;
DIG_D6_D06_042: starting sequence A2 with mutations Y52H, Q140H;
DIG_F4_F04_064: starting sequence A2 with mutations R98G, S129F, D135E, V192D;
DIG_D3_D03_039: starting sequence A2 with deletion of positions 123-132.

### Example IV

### Protein Stability-Based Biosensors as a Screening Platform for New Small Molecule Therapeutics

Many diseases are caused by too little or excess activity of proteins like enzymes and signaling proteins. Screening for small molecule compounds that modulate protein function in a high-throughput fashion is difficult and costly. To address this issue, the present disclosure engineers destabilized variants of disease-related proteins, and then screens for small molecules that stabilize the protein. It can be expected that many compounds that bind tightly enough to stabilize a protein will modulate its activity. For example, small molecules that bind tightly to an enzyme will often bind to its active site, and such molecules could function as competitive inhibitors to enzyme function. Such molecules are highly desirable as, e.g., inhibitors of kinases that function excessively in tumor cells. Many variants of one or multiple enzymes can be analyzed in a multiplexed fashion using DNA barcoding. According to one aspect, many barcoded variants of single or multiple proteins could be assayed in a single well of a microtiter plate, where each well is treated with a different drug candidate, so that tens- or hundreds of thousands of compounds could be rapidly and accurately screened for activity. Using the transcription factor (TF)-biosensor format system, conditionally stable LBD variants could produce mRNA or cDNA that could be sequenced and quantified in comparison to untreated controls to determine degrees of stabilization for each variant and small molecule pair.

Specifically, each member of a panel of enzymes known to be overexpressed in human tumors (see Poliakov, E., Managadze, D. & Rogozin, I. B. Generalized portrait of cancer metabolic pathways inferred from a list of genes overexpressed in cancer. Genetics research international 2014, 646193, doi:10.1155/2014/646193 (2014)), such as EEF1A1, GAPDH and PKM2, as an LBD will be fused in a biosensor construct. Destabilizing mutations will then be screened, which can be quantified using a fluorescent signal as described, or by fusing the LBD to a TF or polymerase to produce mRNA or cDNA and performing next-generation or Sanger sequencing of barcoded transcripts produced by the reporter. Biosensors will be assayed in vivo in microbes, and also using the cell-free system described in EXAMPLE II above. Each well of a microtiter plate will contain the panel of LBD variants (in vivo or cell free). One set of plates will be treated with small molecule drug candidates, and an identical set will be treated with control vehicle. Since microtiter plates have up to 1536 wells, hundreds of thousands of compounds can be screened against hundreds of LBD variants using available automated screening facilities. Ligand-LBD pairs showing stabilization (or destabilization) over control as read out by fluorescence or transcription can then be easily identified and the ligands can become leads for drug development against their cognate LBDs. Further, since multiple LBD variants for a given disease-related protein can be simultaneously assayed, information about which mutational variants are stabilized by which ligands can provide more detailed information about small molecule-protein interactions and protein mechanism, and can potentially contribute to personalizing drug treatments towards classes of disease-related protein mutants.

As an alternative embodiment to this screening protocol, wild-type or known disease-related protein sequences, rather than engineered destabilized protein sequences as above, can be used as LBDs. In this embodiment, the screen will provide information about the following:

a. Which small molecules may stabilize or destabilize the structure of wild-type proteins or disease-related protein mutants.

b. Which small molecules may aid or interfere with the folding process of wild-type proteins or disease-related protein mutants.

### Example V

### Preliminary Engineering of Conditionally Destabilized LBD Biosensors in E. coli

### Experimental Design

This experiment was conducted as a preliminary step to demonstrate the feasibility of engineering conditionally destabilized biosensors in prokaryotes according to certain embodiments as herein disclosed. Plasmids were created each containing a ligand binding domain (LBD) fused to a repressor protein, a GFP reporter regulated by a promoter containing a repressor binding site, and a constitutive promoter expressing mCherry to allow for normalization to account for plasmid copy number differences. Four repressors were tested: LexA, LacI, 933W, and cI from Lambda along with four LBDs: the wildtype digoxin and progesterone binders (See C. E. Tinberg et al., Nature 501, 212, Sep. 12, 2013) and previously described LBD biosensors for digoxin and progesterone (see Feng et al., Elife 4, 1-30 (2015). For each of these constructs, multiple versions were created which varied both in the promoter strength of the repressor-biosensor fusion protein and in the strength of the repressor binding site.

The plasmids were transformed into E. coli and strains were grown in LB-Lennox media with plasmid selective antibiotics and the small molecule drug target digoxin or progesterone. Cultures were grown overnight at 37° C. and diluted 1:100 into fresh media containing the same antibiotics and drug target. Cells were incubated for two more hours at 37° C. before being diluted 1:1 into fresh media containing antibiotics and drug target and immediately analyzed by flow cytometry.

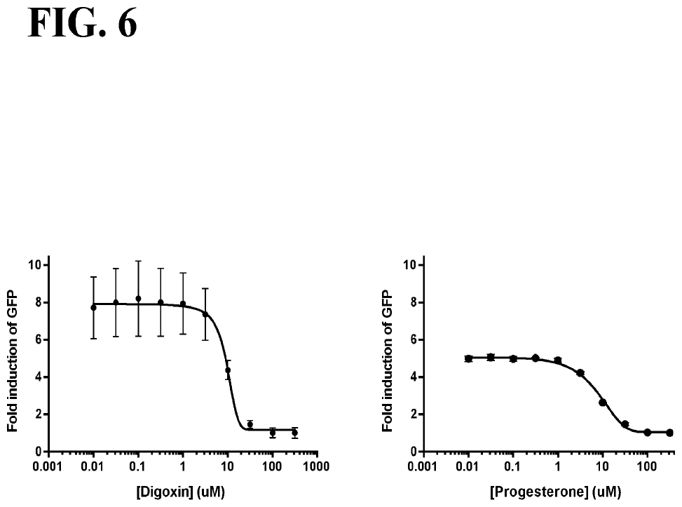

Ligand-dependent sensitivity with a number of the fusion constructs were observed, with the best being the LBDs fused to the N-terminus of the 933W repressor protein (FIG. 6). The GFP/mCherry ratio exhibited an 8-fold change from the de-repressed state (absence of the stabilizing ligand) to the repressed state (presence of the stabilizing ligand) for the best digoxin biosensor (incorporating the DIG1 digoxin-binding LBD from the yeast G-DIG₁-V biosensor), and a 5-fold change for the best progesterone biosensor (incorporating the PRO₁ progesterone-binding LBD from the G-PRO₁-V yeast biosensor) (FIG. 6). This change in repressor stability may result from aggregation of misfolded proteins as the LBD is translated through the ribosomes, degradation through prokaryotic protein quality control machinery, or a combination of both. These results indicate a strong starting point for engineering conditionally destabilized LBD biosensors in E. coli and other prokaryotes.

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

From the foregoing, it will be appreciated that although specific examples have been described herein for purposes of illustration, various modifications may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

What is claimed is:

1. A biosensor comprising a ligand binding domain (LBD) or its variant, wherein the stability of the LBD or its variant is conditioned on the presence of specific small molecule ligands, and wherein the LBD or its variant is fused to a reporter protein.

2. The biosensor of claim 1, wherein the reporter protein comprises a fluorescent protein, a polymerase, a transcription factor (TF), an enzyme, a signaling protein, or a functional protein.

3. The biosensor of claim 2, wherein the TF comprises a transcriptional activator or repressor.

4. The biosensor of claim 1, further comprising elements suitable for screening of specific small molecule ligands that bind and stabilize/destabilize the LBD or its variant in prokaryotic or eukaryotic cells.

5. The biosensor of claim 4, wherein in the absence of a stabilizing ligand the LBD-reporter or the LBD variant-reporter fusion is unstable and degraded or aggregated in the prokaryotic or eukaryotic cells, thereby preventing the reporter protein from carrying out its function.

6. The biosensor of claim 4, wherein in the presence of a stabilizing ligand the LBD-reporter or the LBD variant-reporter fusion is stabilized in the prokaryotic or eukaryotic cells, thereby the reporter protein carries out its function.

7. The biosensor of claim 3, wherein the transcriptional repressor comprises a LexA, Lacl, a 933W, or a cI from Lambda phage.

8. The biosensor of claim 7, wherein in the absence of a stabilizing ligand the LBD-repressor fusion or the LBD variant-repressor fusion is unstable and degraded or aggregated in the prokaryotic or eukaryotic cells, thereby activating transcription of a reporter gene.

9. The biosensor of claim 7, wherein in the presence of a stabilizing ligand the LBD-repressor fusion or the LBD variant-repressor fusion is stabilized in the prokaryotic or eukaryotic cells, thereby abrogating transcription of a reporter gene.

10. The biosensor of claim 1, wherein the LBD or its variant is fused to a RNA polymerase.

11. The biosensor of claim 10, wherein the LBD or its variant is fused to a RNA polymerase omega subunit and a DNA binding domain (DBD).

12. The biosensor of claim 11, wherein the DBD is LexA, LacI, 933W or cI that can activate transcription using the DBD cognate promoter.

13. The biosensor of claim 1, wherein the LBD or its variant is fused to a sigma factor as a sequence-specific transcriptional activator.

14. The biosensor of claim 2, wherein the TF comprises a DNA-binding domain and transcriptional activation domain.

15. The biosensor of claim 14, wherein ligand-induced stabilization of the LBD-TF fusion or the LBD variant-TF fusion activates expression of a reporter gene.

16. The biosensor of claim 15, wherein addition of a cognate ligand stabilizes the LBD-TF fusion or the LBD variant-TF fusion and increases in vivo levels of the TF, thus coupling transcriptional activation to the level of the small molecule ligand.

17. The biosensor of claim 1, wherein the LBD and the reporter protein are genetically fused.

18. The biosensor of claim 1, wherein the LBD and the reporter protein are fused together post-translationally.

19. The biosensor of claim 1, wherein the LBD comprises proteins, enzymes, engineered monoclonal antibodies (mAbs), or mAb fragments, FAbs, scFvs or nanobodies.

20. The biosensor of claim 19, wherein the LBD enzyme comprises EEF1A1, GAPDH or PKM2.

21. A cell-free biosensing system comprising a biosensor comprising a ligand binding domain (LBD) or its variant, wherein the stability of the LBD or its variant is conditioned on the presence of specific small molecule ligands, and wherein the LBD or its variant is fused to a reporter protein.

22. The cell-free biosensing system of claim 21, wherein the reporter protein comprises a fluorescent protein, a polymerase, a transcription factor (TF), an enzyme, a signaling protein, or a functional protein.

23. The cell-free biosensing system of claim 21, wherein the LBD, its variant and the reporter protein are purified or in vitro transcribed and translated using whole cell lysate from cells including bacteria, yeast, human, wheat germ or rabbit reticulocytes.

24. The cell-free biosensing system of claim 22, wherein the TF comprises a DNA-binding domain and transcriptional activation domain.

25. The cell-free biosensing system of claim 24, wherein ligand-induced stabilization of the LBD-TF fusion or the LBD variant-TF fusion activates expression of a reporter gene.

26. The cell-free biosensing system of claim 25, wherein addition of a cognate ligand stabilizes the LBD-TF fusion or the LBD variant-TF fusion and increases levels of the TF, thus coupling transcriptional activation to the level of the small molecule ligand.

27. The cell-free biosensing system of claim 21, wherein the LBD or its variant is fused to the DNA-binding domain of Gal4 and a VP16 activation domain.

28. The cell-free biosensing system of claim 21, wherein the LBD or its variant is fused to an RNA polymerase.

29. The cell-free biosensing system of claim 28, wherein the RNA polymerase comprises T7, T3 or SP6 RNA polymerases.

30. The cell-free biosensing system of claim 21, wherein the LBD comprises proteins, enzymes, engineered monoclonal antibodies (mAbs), or mAb fragments, FAbs, scFvs or nanobodies.

31. The cell-free biosensing system of claim 30, wherein the LBD enzyme comprises EEF1A1, GAPDH or PKM2.

32. A method of screening protein stabilizing small molecule ligand comprising
contacting a sample suspected of containing the small molecule ligand with a biosensor comprising a ligand binding domain (LBD) or its variant, wherein the stability of the LBD or its variant is conditioned on the presence of specific small molecule ligands, and wherein the LBD or its variant is fused to a reporter protein,
detecting the amount of the reporter protein wherein the amount of the reporter protein is dependent on the stability of the LBD or its variant, and
selecting the small molecule ligand that stabilizes the LBD or its variant.

33. The method of claim 32, wherein the reporter protein comprises a fluorescent protein, a polymerase, a transcription factor (TF), an enzyme, a signaling protein, or a functional protein.

34. The method of claim 32, wherein the LBD comprises proteins, enzymes, engineered monoclonal antibodies (mAbs), or mAb fragments, FAbs, scFvs or nanobodies.

35. The method of claim 34, wherein the LBD enzyme comprises EEF1A1, GAPDH or PKM2.

36. The method of claim 32, wherein a library of mutational LBD variants is created.

37. The method of claim 32, wherein sequence of the mutational LBD variant can be determined by sequencing.

38. The method of claim 37, wherein the sequencing is next-generation sequencing.

39. The method of claim 37, wherein the sequencing is Sanger sequencing.

40. The method of claim 32, further comprising selecting for ligand-LBD pairs showing stabilization or destabilization over control.
